(19) United States
(12) Patent Application Publication
(10) Pub. No.: US 2012/0303119 A1
(43) Pub. Date: Nov. 29, 2012

(54) CAPSULAR OPACIFICATION BLOCKING LENS

(75) Inventors: Wayne B. Callahan, Abingdon, VA (US); Paul S. Koch, Warwick, RI (US); Anna S. Hayes, Newton Centre, MA (US); Robert E. Kellan, Methuen, MA (US); Joseph D. Callahan, Abingdon, VA (US); Charles W. Nutter, Bristol, TN (US)

(73) Assignee: Anew Optics, Inc., Bristol, TN (US)

(21) Appl. No.: 13/487,997

(22) Filed: Jun. 4, 2012

Related U.S. Application Data
(63) Continuation-in-part of application No. 13/361,688, filed on Jan. 30, 2012.
Application No. 13/361,688 is a continuation-in-part of application No. 13/113,975, filed on May 23, 2011.
Application No. 13/113,975 is a continuation-in-part of application No. 12/626,459, filed on Nov. 25, 2009.
Application No. 12/626,459 is a continuation-in-part of application No. 12/626,473, filed on Nov. 25, 2009.
(60) Provisional application No. 61/500,203, filed on Jun. 23, 2011.
(60) Provisional application No. 61/437,291, filed on Jan. 28, 2011.
(60) Provisional application No. 61/381,784, filed on Sep. 10, 2010.
(60) Provisional application No. 61/347,083, filed on May 21, 2010.
(60) Provisional application No. 61/118,076, filed on Nov. 26, 2008.
(60) Provisional application No. 61/492,900, filed on Jun. 3, 2011.

(51) Int. Cl. A61F 2/16

(52) U.S. Cl. 623/6.43

(57) ABSTRACT

The invention is directed to a lens that comprises an optic and two haptic rings, one positioned to rest against the posterior capsule distally outward from the optic zone, the other to rest on the anterior capsule some distance from the equator. The haptic rings of the lens are connected by segments of haptic material that may be arched or straight, and sections of open space to provide for ample circulation of the aqueous humor. The optic is positioned against the posterior capsule as close as possible to the nodal zone of the eye.

### REFERENCE TO RELATED APPLICATIONS

This application is a continuation-in-part of U.S. application Ser. No. 13/361,688 filed Jan. 30, 2012, which claims priority to U.S. Provisional Application No. 61/500,203 filed Jun. 23, 2011 and U.S. Provisional Application No. 61/437,291 filed Jan. 28, 2011, and U.S. application Ser. No. 13/113,975 filed May 23, 2011, which claims priority to U.S. Provisional Application No. 61/381,784 filed Sep. 10, 2010 and U.S. Provisional Application No. 61/347,083 filed May 21, 2010, which is a continuation-in-part of U.S. application Ser. No. 12/626,459 filed Nov. 25, 2009 and U.S. application Ser. No. 12/626,473 filed Nov. 25, 2009, which each claim priority to U.S. Provisional Application No. 61/118,076 filed Nov. 26, 2008, and claims priority to U.S. Provisional Application No. 61/492,900 filed Jun. 3, 2011.

### BACKGROUND

1. Field of the Invention

This invention is directed to intraocular lens and haptic devises, to methods for replacement of a natural or artificial lens with an intraocular lens of the invention and, in particular, to intraocular haptic rings that block opacification.

2. Description of the Background

The natural lens of the human eye possesses certain features that are critical to the importance of the inventive lens. The natural eye capsule is generally ovate in shape when seen from the side, and essentially circular in shape when viewed through the cornea. The imputed radius of curvature of the anterior capsule of the lens is greater than the imputed radius of curvature of the posterior capsule, and though the ratio of the radii of curvature changes if the eye is measured in a distance vision state or in a near vision (or accommodative) state, the absolute arc length of the anterior and posterior capsule segments do not change.

The capsule is retained in the eye by means of a network of zonules, which are fixed length fibers that do not demonstrate elasticity, and are attached to the capsule at various points anterior to, posterior to and within the fornix (or equatorial zone) of the eye, and at the other end to the ciliary body at certain point(s) posterior to the ciliary point. When the ciliary body expands anteriorly and toward the pupil, the zonules relax tension on the capsule and the capsule becomes more circular in shape, thus providing a greater diopter power for near vision accommodation. When the ciliary body retracts posteriorly and away from the pupil, the zonules exert force on the capsule, stretching the capsule outward and making a flatter ovate shape, thus providing for distance vision. The natural lens capsule at the equator varies in diameter between approximately 8.9 millimeters in the accommodative state to approximately 9.5 millimeters in the distance state. All measurements are approximate as each eye varies in size and capsular dimensions can change over time, particularly when considering the juvenile eye.

While the angles of the zonules to the ciliary point are most variable in accommodation in the most anterior and posterior sets of zonules, the zonules closest to the fornix are likely to demonstrate least change in accommodation thus greatest stability in maintaining the correct position of the capsule in the eye.

A third set of zonules attaches the ciliary body to a series of points through the vitreous. It is believed that the third set of zonules is fundamental in maintaining correct positioning of the capsule in the eye and preventing excess anterior dislocation of the capsule in accommodation.

Most intraocular lenses currently available are essentially two-dimensional, and consist of an optic centrally located between plate, c-loop, or other haptics and with overall lens diameter of approximately 11 millimeters. The lens diameter is, notably, longer than the natural diameter of the lens capsule, as the purpose of these conventional lens designs is to stretch the capsule to an essentially flattened shape. Flattening the capsule can have significant negative consequences. Firstly, flattening pulls the posterior capsule forward in the eye which may create negative pressure on the vitreous and increase the risk of detachment of the retina. Secondly, these lenses generally do not have haptics that preserve the circular configuration of the capsule at the fornix, which means that the relationship between the capsule and the zonules is disrupted which may cause undue stress on the zonules and on the ciliary body. Thirdly, these lenses generally cause the capsule to fibrose over time, and those sections of the capsule that are not separated by the lens tend to adhere to each other. Fourthly, contact between the capsule and the lens, both anterior and posterior, predisposes the eye to develop anterior and posterior capsular opacification which can ultimately cloud the optical area of the lens and, without additional surgery, diminish the eyesight of the patient. After cataract surgery within the capsule that contained the natural lens are cells similar to the cells that initially grew the cataractous condition. The cells migrate from along the anterior surface of the lens to the equator where they accumulate with additional cells formed at the equator and mass with them to form a blanket, then move posteriorly along the natural lens capsule until the density is such that light traveling through the lens is impeded impairing the patient's vision. Laser light is used to split the posterior portion of the capsule to allow the patient's vision to be restored.

Most intraocular lenses have optics that range in diameter from 5 millimeters to 6 millimeters; in rare occasions are lens optics greater than 6 millimeters, as the greater diameter tends to cause too much bulk to allow insertion of the lens into the eye through an incision of less than 3 mm. Because the pupil of the eye is not centered in the eye but located approximately 5° toward the nose, some intraocular lens recipients complain that they have a “blind spot” which is generally thought to result from the patient perceiving the edge of the optic, particularly with lenses with optics of 5 mm or less. This condition can become exacerbated by the position of the lens in the eye; the farther anterior the lens is positioned, the greater the distance from the lens to the retina, therefore the higher risk that the edge of the lens could be visible, especially when the pupil is dilated (such as when driving at night).

Thus, a need exists for an intraocular lens that is less bulky, provides an improved depth of field, diminishes risks of blind spots, can be inserted into the eye through a small incision, and provides for accommodative correction, and yet preserves the natural configuration of the eye capsule.

### SUMMARY

The present invention overcomes the problems and disadvantages associated with current strategies and designs, and provides new tools and methods for lens replacement therapies.

One embodiment of the invention is directed to an intraocular lens. The lens comprises a haptic that contains an anterior ring adapted to couple to the natural lens capsule of a human eye at a distance anterior to a fornix of the capsule, a posterior ring adapted to couple to the posterior of the capsule at a distance posterior to the fornix of the capsule, a haptic segment coupling the anterior ring and posterior ring, and an optic centered posterior to the posterior ring so as to be positioned at the deepest natural point of the lens capsule in the eye.

Preferably the lens is comprised of a hydrophilic acrylic. The hydrophilic acrylic is preferably a silicone, PMMA, a polymer or a combination thereof. In the preferred embodiment, the haptic and optic are manufactured as a single unit. Preferably the lens is comprised of at least two materials. Preferably, the optic is at least 6 mm in diameter or the optic is at maximum 6 mm in diameter.

In the preferred embodiment, the anterior ring is equal to or greater in diameter than the posterior ring. Preferably, the posterior ring is equal to or greater in diameter than the anterior ring. The lens preferably further comprises at least one easement coupling the anterior ring to the haptic segment. The lens preferably further comprises at least one easement coupling the posterior ring to the haptic segment. The haptic segment is preferably one of straight or arched. The haptic segment is preferably one of perforated or solid. Preferably, when implanted in the eye, the anterior ring rests both anterior to and within the fornix of the capsule. Preferably, the optic is suspended between the anterior and posterior rings.

Another embodiment of the invention is directed to a method of replacing an existing lens of an eye comprising removing the existing lens from the eye, and inserting into the eye the lens described herein.

Another embodiment of the invention is directed to a method of preventing or ameliorating capsular opacification. The method comprises placing the intraocular lens described herein into the capsule of an eye of a patient.

Preferably the intraocular lens blocks cell migration and prevents posterior capsular opacification. Preferably, the intraocular lens blocks cell migration and prevents anterior capsular opacification. In the preferred embodiment, the intraocular lens does not substantially alter the natural configuration of the capsule.

Other embodiments and advantages of the invention are set forth in part in the description, which follows, and in part, may be obvious from this description, or may be learned from the practice of the invention.

### DESCRIPTION OF THE FIGURES

FIG. 1 depicts one embodiment of the lens of the invention from the top and side views.

FIG. 2 depicts the lens of FIG. 1 as viewed from the top.

FIG. 3 depicts one embodiment of the lens of the invention from the side view.

FIG. 4 depicts the lens of FIG. 3 from the side view.

### DESCRIPTION OF THE INVENTION

The lens of the invention addresses key considerations within the field of ophthalmology and specifically within the field of lens replacement surgery, whether for cataracts, presbyopia correction, or other mechanism. The invention addresses the importance of maintaining as much as possible the natural configuration of the capsule, and in particular the configuration of the posterior capsule. The invention addresses the importance of maintaining a distance between the anterior capsule and the anterior of the optic.

The invention is directed to a lens that comprises an optic and two or more haptic rings, one positioned to rest against the posterior capsule distally outward from the optic zone, the other to rest on the anterior capsule some distance from the equator. The haptic rings of the lens are connected by segments of haptic material that may be arched or straight, and sections of open space to provide for ample circulation of the aqueous humor. In the first instance, the anterior ring is located anterior of the fornix and the fornix does not contact the haptic ring but is held in place by the relationship between the anterior and posterior haptic rings. In the second instance, the anterior haptic ring is positioned to rest in the fornix, with its most anterior portion anterior of the fornix but with the remainder of the haptic ring in contact with the fornix so as to prevent any stress on any section of the zonules and maintain the configuration of the fornix. Preferably the haptic rings are the same size, although the anterior ring may be larger than the posterior ring or the posterior ring may be larger than the anterior ring. The haptic rings may be solid, or they may be hollowed or indented on the interior and/or exterior surfaces to minimize the bulk of the lens and provide for some flexibility to adjust to different capsular sizes. A function of the haptic rings is to keep the capsule configured as naturally as possible, while the anterior ring is designed to arrest the migration of lens epithelial cells along the anterior capsule to the fornix, thereby mitigating the onset or occurrence of Anterior Capsule Opacification (ACO), and the posterior ring is positioned on the posterior capsule to prevent incursion of lens cortical material into the optical zone in the form of PCO (Posterior Capsular Opacification). The haptic perforations are designed to enhance circulation of the aqueous humor throughout targeted areas of the capsule so as to preserve overall capsular hydration and health.

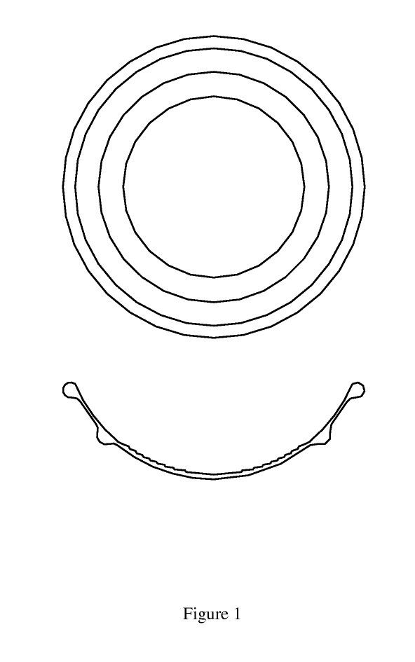

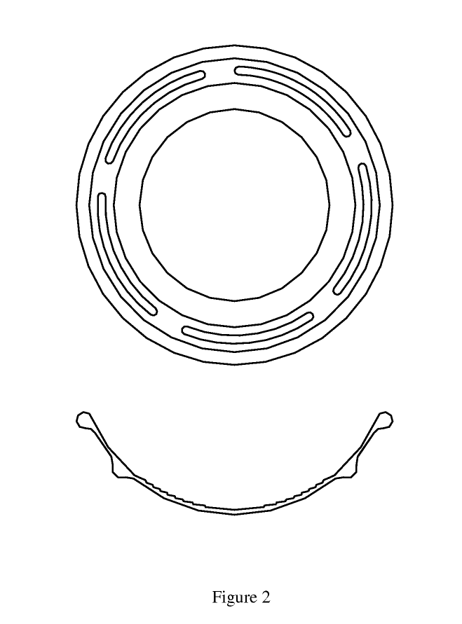

The lens of the invention blocks cell migration and prevents posterior capsular opacification. The lens preferably has an anterior surface (FIGS. 1 and 2) with rings and a posterior surface. The anterior surface preferably has an anterior ring that prevents or blocks lens epithelial cells from migrating along the anterior portion of the natural lens toward the capsular equator. The posterior surface of the lens preferably has a posterior ring that blocks lens cortical material formed in the equatorial zone from passing into the optical zone of the posterior capsule. Between the anterior and posterior ring preferably there are perforations, slots or spaces cut into the lens to allow aqueous flow into the desired areas of the capsule. The aqueous flow within the eye allows cells and cortical fibroblasts that are arrested by the anterior and posterior rings to be flushed from the capsule and carried out of the eye through the trabecular meshwork by the natural fluid flow system of the anterior segment of the eye.

The optic is preferably designed to be positioned as far posterior in the capsule as possible, resting against the posterior capsule and retained in position by the posterior haptic ring. The functionality of the location of the optic is important in at least three respects: first, the posterior capsule retains its natural configuration as much as possible, which preserves positive pressure on the vitreous and mitigates some of the risks of retinal detachment; second, the optic is positioned as close as possible to the nodal zone of the eye, which provides for greatest optical depth of field, thus enhancing the patient's quality of vision; third, the location of the optic and the diameter of the optic mitigate significantly any potential risk that the patient's vision will be disrupted by being able to discern the edge of the optic.

The entire lens, haptic and optic assembly, is configured and designed so as to be able to be inserted in the human eye through an incision of less than 3 mm. Lens replacement surgery is preferably performed using very small incisions, reducing thereby the trauma to the patient and mitigating the need for sutures.

A preferred embodiment of the invention is directed to an intraocular lens with an ultra thin optic of less than 475 microns at its thickest point and an optic diameter of 6 millimeters or greater.

Another preferred embodiment of the invention is directed to a pair (or more, e.g., three or four) of haptic rings positioned to rest against the anterior capsule, the fornix, the posterior capsule or any combination of these so as to maintain capsule configuration especially of the posterior capsule, and mitigate the onset and severity of any capsular opacification.

Another preferred embodiment of the invention is directed to an arch in the haptic segment between the anterior and posterior rings to create flexibility and respond do differences in capsular size.

Another preferred embodiment of the invention is directed to perforations or open spaces in the haptic segments to provide for circulation of the aqueous humor in desired areas of the capsule so as to mitigate the onset of capsular fibrosis, adhesions, and atrophy, and cleanse the eye of any detached lens epithelial cells or cortical material.

Another preferred embodiment of the invention is directed to placement of the optic within the lens capsule at the posterior capsule as close to the nodal zone of the eye as possible.

The following examples illustrate embodiments of the invention, but should not be viewed as limiting the scope of the invention.

### EXAMPLES

FIG. 1 depicts one embodiment of the lens of the invention from the top and side views, demonstrating the anterior and posterior rings and the location of the inventive optic posterior and central to the posterior ring such that the posterior haptic rests against the posterior capsule at some distance from the capsular equator, or fornix, and the posterior optic is positioned as far back in the capsule as possible, resting against the center of the posterior capsular optic zone. In this figure the relative dimensions of the anterior and posterior rings may be altered so that the radius of the posterior ring may be smaller than, equal to, or greater than the radius of the anterior ring. The anterior ring may be positioned anterior to the fornix, or may be located in the fornix of the capsule. FIG. 1 also depicts a haptic segment between the anterior and posterior haptic rings that is essentially planar and that may be configured as a solid plate or contain perforations of various sizes for increased flexibility and hydration of the capsule. The dimensions of the haptic segments may be altered depending upon the relationship between the anterior and posterior rings so as to preserve overall capsular dimensions.

FIG. 2 depicts the lens of FIG. 1 as viewed from the top, delineating a possible configuration of haptic perforations for hydration and flexibility.

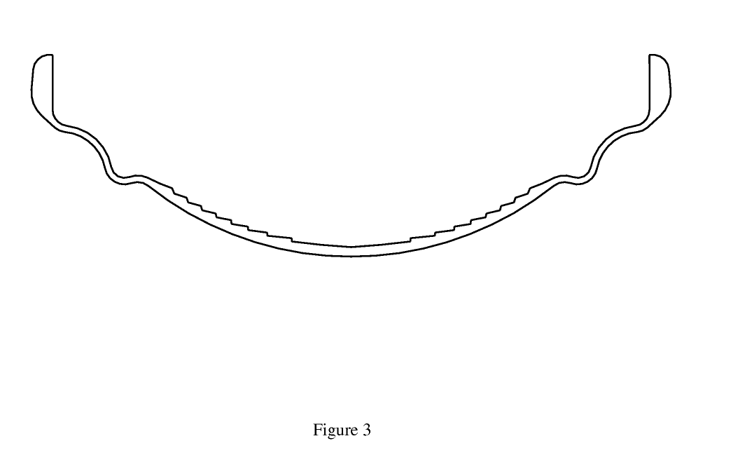

FIG. 3 depicts one embodiment of the lens of the invention from the side view demonstrating an anterior haptic ring that rests in the fornix and extends anteriorly to the anterior capsule, so as to conform to the configuration of the fornix of the natural lens capsule. In this figure the relative dimensions of the anterior and posterior rings may be altered so that the radius of the posterior ring may be smaller than, equal to, or greater than the radius of the anterior ring. The haptic segment between the anterior and posterior rings is preferably arched to provide flexibility for sizing the lens to capsules of different overall dimensions. Preferably, the distance between the haptic rings and the angle of curvature of the arched haptic segment may be altered to conform to the overall desired dimension of the lens and the relationship between the haptic rings. FIG. 3 also demonstrates a preferred configuration of the posterior ring in which the ring is indented on its inner surface so as to increase flexibility and reduce overall bulk. Preferably, the anterior ring is configured with either or both inside and/or outside easements, either vertical or horizontal, to provide for greater flexibility of the anterior ring to adjust to different capsular dimensions, while preserving the suitable degree of tension to ensure accurate position in the eye and centration and consistent location of the optic at the posterior of the capsule.

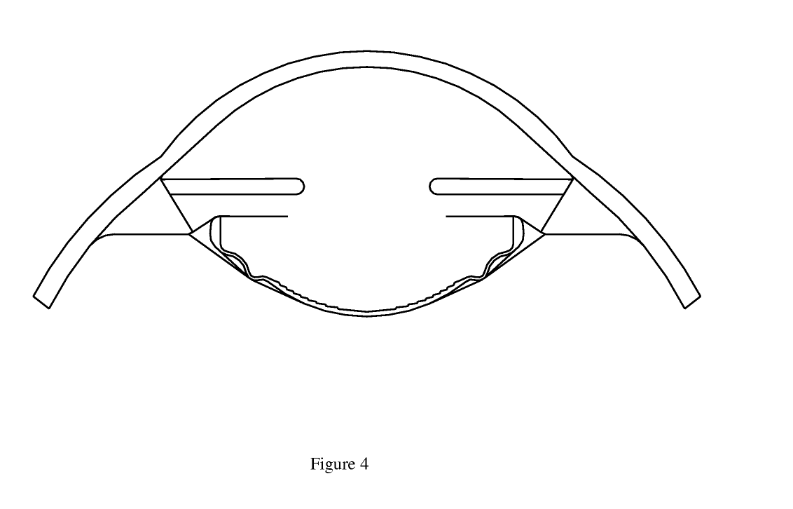

FIG. 4 depicts the lens of FIG. 3 from the side view locating the lens within the natural capsule of the eye, demonstrating the preferred position of the lens within the eye and the anterior capsule with capsulorhexis as typical for lens replacement surgery.

Other embodiments and uses of the invention will be apparent to those skilled in the art from consideration of the specification and practice of the invention disclosed herein. All references cited herein, including all publications, all U.S. and foreign patents and patent applications, are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. An intraocular lens comprising a haptic that contains:
an anterior ring adapted to couple to the natural lens capsule of a human eye at a distance anterior to a fornix of the capsule;
a posterior ring adapted to couple to the posterior of the capsule at a distance posterior to the fornix of the capsule;
a haptic segment coupling the anterior ring and posterior ring; and
an optic centered posterior to the posterior ring so as to be positioned at the deepest natural point of the lens capsule in the eye.

2. The lens of claim 1, which is comprised of a hydrophilic acrylic.

3. The lens of claim 2, wherein the hydrophilic acrylic is a silicone, PMMA, a polymer or a combination thereof.

4. The lens of claim 1, wherein the haptic and optic are manufactured as a single unit.

5. The lens of claim 4, wherein the lens is comprised of at least two materials.

6. The lens of claim 1, wherein the optic is at least 6 mm in diameter

7. The lens of claim 1, wherein the optic is at maximum 6 mm in diameter.

8. The lens of claim 1, wherein the anterior ring is equal to or greater in diameter than the posterior ring.

9. The lens of claim 1, wherein the posterior ring is equal to or greater in diameter than the anterior ring.

10. The lens of claim 1, further comprising at least one easement coupling the anterior ring to the haptic segment.

11. The lens of claim 1, further comprising at least one easement coupling the posterior ring to the haptic segment.

12. The lens of claim 1, wherein the haptic segment is one of straight or arched.

13. The lens of claim 1, wherein the haptic segment is one of perforated or solid.

14. The lens of claim 1, wherein, when implanted in the eye, the anterior ring rests both anterior to and within the fornix of the capsule.

15. The lens of claim 1, wherein the optic is suspended between the anterior and posterior rings.

16. A method of replacing an existing lens of an eye comprising removing the existing lens from the eye, and inserting into the eye the lens of claim 1.

17. A method of preventing or ameliorating capsular opacification comprising: placing the intraocular lens of claim 1 into the capsule of an eye of a patient.

18. The method of claim 17, wherein the intraocular lens blocks cell migration and prevents posterior capsular opacification.

19. The method of claim 17, wherein the intraocular lens blocks cell migration and prevents anterior capsular opacification.

20. The method of claim 17, wherein the intraocular lens does not substantially alter the natural configuration of the capsule.
